(19) United States
(12) Patent Application Publication
(10) Pub. No.: US 2026/0272385 A1
(43) Pub. Date: Sep. 17, 2026

(54) Sensor Device

(71) Applicant: Taiyo Yuden Co., Ltd., Tokyo (JP)

(72) Inventors: Hiroki HORIUCHI, TOKYO (JP); Katsuhiro OYAMA, TOKYO (JP); Yuki SAITO, TOKYO (JP)

(21) Appl. No.: 19/567,842

(22) Filed: Mar. 16, 2026

(30) Foreign Application Priority Data
Mar. 17, 2025 (JP) 2025-042961

(51) Int. Cl. A61B 5/00; A61B 5/145; A61B 5/1455

(52) U.S. Cl. CPC A61B 5/6843; A61B 5/14532; A61B 5/1455; A61B 5/6824; A61B 5/6831

(57) ABSTRACT

A sensor device includes a light-emitting element, a prism that is of a trapezoidal contour as viewed along a direction and has a first bottom surface for contact with a surface of a human body, a slanted surface to which light emitted from the light-emitting element is applied, and a second bottom surface lying parallel to the first bottom surface, a light-detecting element that detects light that has been reflected by a region in the human body, transmitted through the prism, and emitted from the second bottom surface, an estimating section that estimates a concentration of a biological component contained in the human body from an output from the light-detecting element, a pressing mechanism that presses the prism against the human body, and a controlling section that controls the pressing mechanism.

### BACKGROUND

The present disclosure relates to a sensor device.

In the fields of medical care and health care, it has been customary to examine a concentration of a biological component contained in human bodies, e.g., the concentration of glucose in blood, by taking blood samples from the human bodies. However, taking blood samples from patients occurs with concerns about physical and mental burdens such as pain, discomfort, and anxiety of the patients and concerns about infectious diseases.

Consequently, research efforts have been made to use light such as near-infrared light to obtain a noninvasive estimate of the concentration of a biological component (see, for example, JP 2016-112042A and JP 2020-163188A).

### SUMMARY

According to the noninvasive optical measurement technology, the accuracy of measurement varies depending on the pressing force, i.e., the pressing force, applied to the human body.

According to the technology disclosed in JP 2016-112042A, a pressing force applied when an amplitude of a plethysmogram based on light from the human body is maximum is set as an optimum pressing force. This technology is based on the fact that the plethysmogram is varied by blood vessel pulsations.

However, since the technology needs measurement of a plethysmogram, it is necessary to measure light reflected from a blood vessel such as a thick artery located deeply in the human body. For utilizing the technology disclosed in JP2016-112042A in estimating the concentration of a biological component by use of light reflected by a region in the skin of the human body, an optical system for measuring light reflected from a deep region of the human body is required in addition to an optical system for measuring light reflected from a region in the skin.

According to the technology disclosed in JP2020-163188A, a state of contact between a prism and a skin to be measured, i.e., a contact stress and a contact angle, is calculated using a strain sensor. It is then determined whether the measured state of contact has satisfied conditions for securing the desired accuracy of detection or not, i.e., whether the contact stress falls within a predetermined range or not.

However, according to the technology disclosed in JP2020-163188A, the concentration of a biological component is not able to be estimated if the conditions for securing the desired accuracy of detection are not satisfied. In addition, JP2020-163188A does not disclose a function to automatically control the pressing force adequately.

It is desirable to provide a sensor device that is capable of estimating the concentration of a biological component contained in a human body and automatically controlling a pressing force by using an optical system for measuring light reflected by a region in the skin of the human body.

In accordance with an aspect of the present disclosure, there is provided a sensor device including a light-emitting element, a prism that is of a trapezoidal contour as viewed along a direction and has a first bottom surface for contact with a surface of a human body, a slanted surface to which light emitted from the light-emitting element is applied, and a second bottom surface lying parallel to the first bottom surface, the prism refracting light emitted from the light-emitting element toward the human body, a light-detecting element that detects light that has been reflected by a region in the human body, transmitted through the prism, and emitted from the second bottom surface, an estimating section that estimates a concentration of a biological component contained in the human body from an output from the light-detecting element, a pressing mechanism that presses the prism against the human body, and a controlling section that controls the pressing mechanism. When a change in the output from the light-detecting element exceeds a first threshold value, the controlling section controls the pressing mechanism to increase its pressing force until the change in the output from the light-detecting element becomes equal to or smaller than the first threshold value, and after the change in the output from the light-detecting element has become equal to or smaller than the first threshold value, the controlling section maintains the pressing force. The estimating section estimates the concentration of the biological component contained in the human body from the output from the light-detecting element under the maintained pressing force.

According to the aspect of the present disclosure, the sensor device estimates the concentration of the biological component by making use of the light reflected by the region in the skin of the human body, using an optical system that includes the light-emitting element, the light-detecting element, and the prism. Moreover, the sensor device also automatically adequately controls the pressing force generated by the pressing mechanism using the output from the light-detecting element of the same optical system.

The controlling section may control the pressing mechanism to reduce the pressing force and then maintain the pressing force after the change in the output from the light-detecting element has become equal to or smaller than the first threshold value.

The controlling section may derive the first threshold value by multiplying a maximum value of the change in the output from the light-detecting element by a predetermined coefficient.

In this case, the controlling section may derive the first threshold value while controlling the pressing mechanism to increase the pressing force.

The controlling section may drive the pressing mechanism in a first mode during an initial stage of its control over the pressing mechanism, and after the initial stage, may drive the pressing mechanism in a second mode that is of a lower rate than the first mode to increase the pressing force from the pressing mechanism until the change in the output from the light-detecting element becomes equal to or smaller than the first threshold value.

In this case, the controlling section may drive the pressing mechanism in the first mode until a change in the output from the light-detecting element per unit time becomes equal to or larger than a second threshold value, and after the change in the output from the light-detecting element per unit time has become equal to or larger than the second threshold value, may drive the pressing mechanism in the second mode.

Alternatively, the controlling section may drive the pressing mechanism in the first mode until the output from the light-detecting element becomes equal to or larger than a third threshold value, and after the output from the light-detecting element has become equal to or larger than the third threshold value, may drive the pressing mechanism in the second mode.

The light-emitting element may emit near-infrared light in a wavelength range of 780 to 2500 nm.

According to the aspect of the present disclosure, the sensor device is capable of both estimating the concentration of a biological component contained in a human body and automatically controlling a pressing force by using an optical system for measuring light reflected by a region in the skin of the human body.

### BRIEF DESCRIPTION OF THE DRAWINGS

FIG. 1 is a front elevational view of a sensor device according to an embodiment of the present disclosure as it is in use on a human body;

FIG. 2 is a cross-sectional view of a part of the sensor device and the human body, illustrating principles of the sensor device for obtaining an estimate of a concentration of a biological component in the human body;

FIG. 3 is a plan view of a part of the sensor device according to the embodiment;

FIG. 4 is a plan view of a part of a sensor device according to a modification of the embodiment;

FIG. 5 is a plan view of a part of a sensor device according to another modification of the embodiment;

FIG. 6 is a block diagram of a control system of the sensor device according to the embodiment;

FIG. 7 is a graph illustrating optical absorption spectrums of oxygenated hemoglobin and reduced hemoglobin;

FIG. 8 is a graph illustrating a relation between pressing forces and outputs from a light-detecting element;

FIG. 9 is a flowchart of a process of using the sensor device according to the embodiment;

FIG. 10 is a flowchart of another process of using the sensor device according to the embodiment; and

FIG. 11 is a flowchart of still another process of using the sensor device according to the embodiment.

### DETAILED DESCRIPTION OF THE PREFERRED EMBODIMENT

A preferred embodiment of the present disclosure will be described in detail below with reference to the accompanying drawings. A scale of the drawings is not necessarily accurate and some features may be illustrated as exaggerated or omitted from illustration.

Sensor devices according to the embodiment of the present disclosure have components mounted on a part, e.g., an arm, of the body of an examinee such as a patient and are used to measure the concentration of a biological component contained in the body of the examinee.

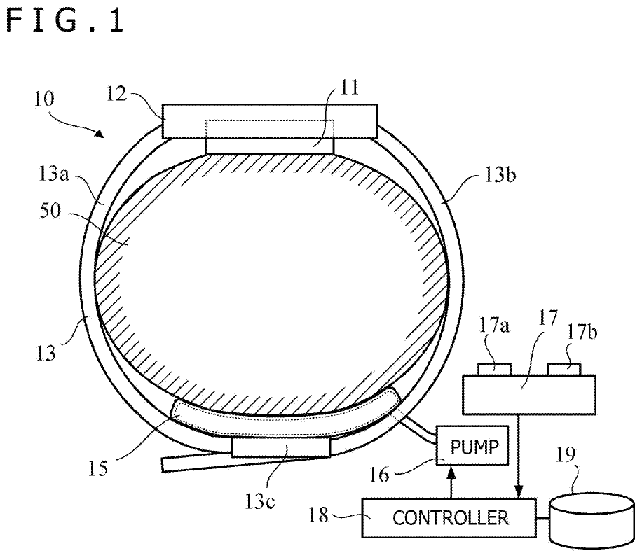

As illustrated in FIG. 1, a sensor device 10 according to the embodiment includes a housing 11, a support 12, a belt 13, an air bag 15, a pump 16, a switch unit 17, a controller 18, and a storage device 19.

The housing 11 accommodates therein an optical system of the sensor device 10 as described later.

The support 12 is shaped as a flat plate that supports the housing 11.

The belt 13 has two belt pieces 13a and 13b and a buckle 13c disposed remotely from the support 12. Each of the belt pieces 13a and 13b has an end and an opposite end, and the support 12 has both ends that are joined to the respective ends of the support 12. The buckle 13c secures the respective opposite ends of the belt pieces 13a and 13b. For example, the buckle 13c may have prongs engaging in holes in the belt pieces 13a and 13b, or the buckle 13c may be fixed to the belt pieces 13a and 13b by a hook-and-loop fastener. In use, the belt 13 is wound around an arm 50 of the examinee such that the optical system of the sensor device 10 that is housed in the housing 11 is placed in a stationary position near the arm 50.

Alternatively, the optical system of the sensor device 10 may be disposed near another region of the examinee, such as one of the four limbs, the neck, the head, or the abdominal part of the examinee, with the belt 13 wound around the other region.

The air bag 15 is sandwiched between the belt 13 and the human body, e.g., the arm 50. The pump 16 delivers air into the air bag 15. The air bag 15 and the pump 16 jointly make up a pressing mechanism for pressing the optical system in the housing 11 against the human body.

Specifically, when the pump 16 is actuated, it delivers air into the air bag 15, expanding the air bag 15. When the air bag 15 is expanded, it exerts a tensile force to the belt 13, causing the support 12 to press the housing 11 toward the arm 50. Therefore, the optical system in the housing 11 is pressed against the arm 50.

The switch unit 17 has a start button 17a and a stop button 17b. When the examinee or a medical attendant in charge presses the start button 17a, the switch unit 17 supplies the controller 18 with a start command, enabling the sensor device 10 to start measuring the concentration of the biological component of the examinee. When the examinee or the medical attendant presses the stop button 17b, the switch unit 17 supplies the controller 18 with a stop command, causing the sensor device 10 to stop measuring the concentration of the biological component of the examinee.

The controller 18 is a computer that operates according to computer programs, for example. The controller 18 is connected to the switch unit 17 through a wireless or wired link for receiving the start command from the switch unit 17. The controller 18 is also connected to the pump 16 through a wireless or wired link for supplying a drive signal to the pump 16. The controller 18 is further connected to the optical system in the housing 11 through a wireless or wired link for supplying a drive signal to the optical system and receiving a measurement signal from the optical system.

As described later, the controller 18 functions as a controlling section for controlling the pressing mechanism and as an estimating section for estimating the concentration of a biological component contained in the human body.

The storage device 19 stores data used by the controller 18 and the computer programs run by the controller 18.

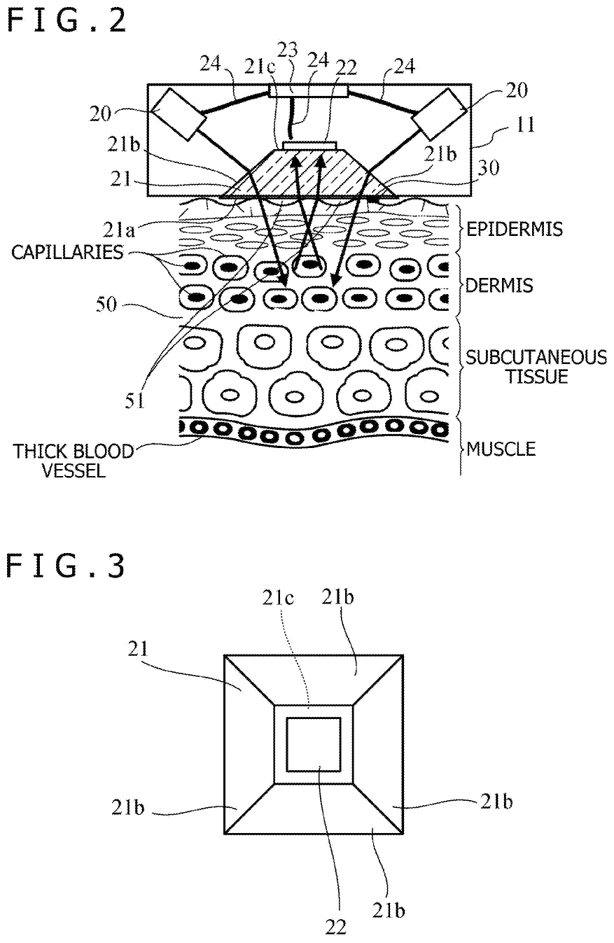

FIG. 2 illustrates the housing 11 in cross section along a plane lying parallel to the sheet of FIG. 1. The housing 11 accommodates the optical system therein as described above. The optical system includes multiple light-emitting elements 20, a trapezoidal prism 21, and a light-detecting element 22. The housing 11 also houses therein a drive circuit 23 that is connected to the light-emitting element 20 and the light-detecting element 22 by wires 24. The drive circuit 23 and the wires 24 are mounted on a flexible printed circuit board, for example.

Each of the light-emitting elements 20 is a semiconductor laser or a light-emitting diode (LED), for example, and emits near-infrared light in a wavelength range of 780 to 2500 nm.

The trapezoidal prism 21 is used to refract the light emitted from the light-emitting elements 20 toward the arm 50 and to transmit therethrough light that has been reflected by a region inside the arm 50. In FIG. 2, thick arrows represent paths of the light in the arm 50 and the trapezoidal prism 21.

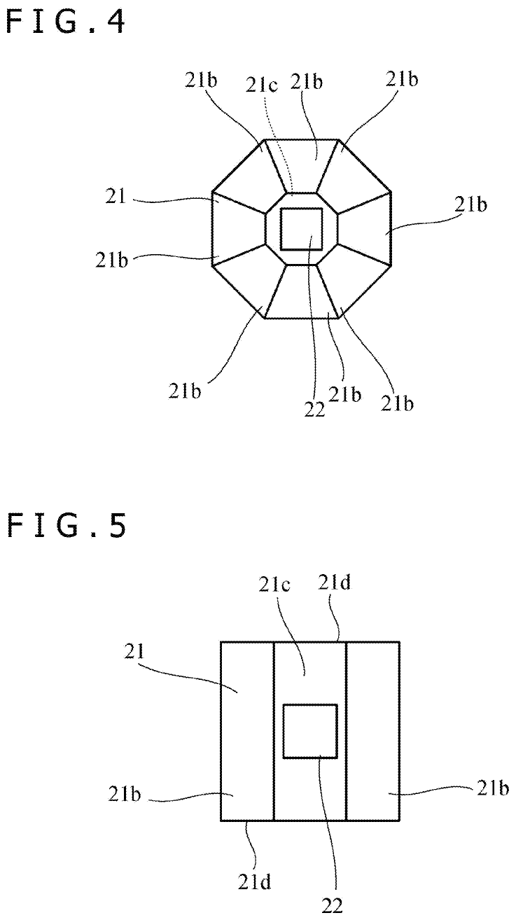

The trapezoidal prism 21 has a trapezoidal, except rectangular, contour as viewed along a certain direction. Specifically, the trapezoidal prism 21 includes two sides parallel to each other and has a certain thickness between the two parallel sides. As illustrated in cross section along a plane in FIG. 2 and viewed along a certain direction perpendicular to the sheet of FIG. 2, the trapezoidal prism 21 has a trapezoidal, except rectangular, contour defined by the two sides and other two sides joining the two sides at their ends. FIG. 3 illustrates, in plan, the trapezoidal prism 21 and the light-detecting element 22 of the sensor device 10 according to the embodiment. FIG. 4 illustrates, in plan, the trapezoidal prism 21 and the light-detecting element 22 of a sensor device 10 according to a modification of the embodiment. FIG. 5 illustrates, in plan, the trapezoidal prism 21 and the light-detecting element 22 of a sensor device 10 according to another modification of the embodiment.

As illustrated in FIGS. 2 and 3, the trapezoidal prism 21 is more accurately shaped as a truncated pyramid. As illustrated in FIGS. 2 and 4, the trapezoidal prism 21 according to the modification is shaped as an octagonal truncated pyramid. The trapezoidal prism 21 may alternatively be shaped as a truncated polygonal pyramid. As illustrated in FIGS. 2 and 5, the trapezoidal prism 21 according to the other modification is shaped as a trapezoidal column.

The trapezoidal prism 21 has a first bottom surface 21a, multiple slanted surfaces 21b, and a second bottom surface 21c. The first bottom surface 21a corresponds to a lower bottom of a trapezoid, and the second bottom surface 21c corresponds to the upper bottom of the trapezoid. The second bottom surface 21c lies parallel to the first bottom surface 21a and is smaller in area than the first bottom surface 21a. The terms “bottom surface,” “lower bottom,” and “upper bottom” refer to geometrical terms of a trapezoid and should not be construed as limiting orientations of the optical system in use.

The slanted surfaces 21b join the first bottom surface 21a and the second bottom surface 21c to each other. The trapezoidal prism 21 shaped as a trapezoidal column illustrated in FIG. 5 further has two side surfaces 21d lying parallel to each other.

As illustrated in FIG. 2, the first bottom surface 21a is held in surface contact with the surface of the arm 50. Although multiple infinitesimal spaces 51 may be present between the first bottom surface 21a and the surface of the arm 50, the first bottom surface 21a is kept in surface contact with the surface of the arm 50 despite those infinitesimal spaces 51.

Each light-emitting element 20 is disposed in facing relation to one slanted surface 21b to apply emitted light to the slanted surface 21b. Another prism, not illustrated, may be interposed between at least one light-emitting element 20 and the slanted surface 21b to which the light from the light-emitting element 20 is applied to adjust the angle of incidence of the light on the slanted surface 21b.

As illustrated in FIGS. 3 and 4, the number of the light-emitting elements 20 may not necessarily be in agreement with the number of the slanted surfaces 21b of the trapezoidal prism 21. However, there may be as many light-emitting elements 20 as the number of the slanted surfaces 21b in facing relation to all the slanted surfaces 21b of the trapezoidal prism 21.

The light-detecting element 22 is a photodiode, for example, for converting the light it has detected into an electric signal. The light-detecting element 22 is disposed in facing relation to the second bottom surface 21c of the trapezoidal prism 21. Of the light emitted from the light-emitting element 20, the light that has been reflected by the region inside the arm 50, transmitted through the trapezoidal prism 21, and left the trapezoidal prism 21 from the second bottom surface 21c is detected by the light-detecting element 22.

The drive circuit 23 energizes the light-emitting element 20 and the light-detecting element 22. The drive circuit 23 selectively activates and inactivates the light-emitting element 20 and the light-detecting element 22 in response to commands that are supplied from the controller 18 through a wireless or wired link. Moreover, the drive circuit 23 transmits an output from the light-detecting element 22 to the controller 18 through a wireless or wired link.

The controller 18 functions as an estimating section for estimating the concentration of a biological component contained in the human body from the output of the light-detecting element 22.

The controller 18 also functions as a controlling section for adjusting the pressing force that the trapezoidal prism 21 applies to the arm 50 through the air bag 15.

As illustrated in FIG. 2, a force sensor 30 is formed on or fixed to the first bottom surface 21a of the trapezoidal prism 21. The force sensor 30 is a piezoelectric sensor or a strain gage sensor, for example, that measures the pressing force that the trapezoidal prism 21 applies to the arm 50. The force sensor 30 is connected to the drive circuit 23. A signal representing a measured result from the force sensor 30 is transmitted from the drive circuit 23 to the controller 18 through a wireless or wired link.

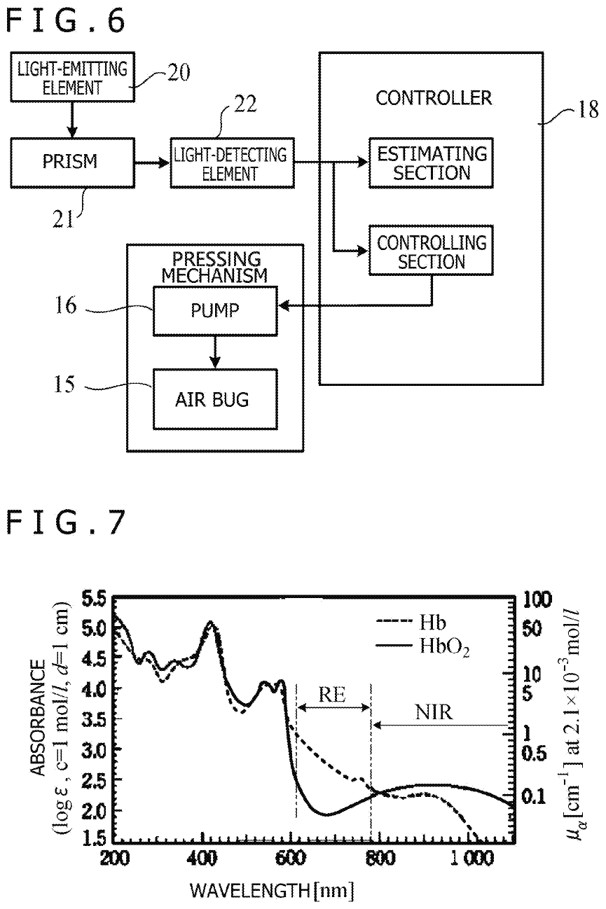

A control system of the sensor device 10 according to the embodiment is illustrated in FIG. 6.

As illustrated in FIG. 2, the light emitted from each light-emitting element 20 is refracted by the slanted surface 21b of the trapezoidal prism 21, further reflected by the first bottom surface 21a and the surface of the arm 50, and travels into the arm 50. Part of the light is absorbed by the dermis in the arm 50 whereas the remainder of the light is reflected back toward the trapezoidal prism 21. Of the reflected light, the light that has traveled to the trapezoidal prism 21 is refracted by the surface of the arm 50 and the first bottom surface 21a of the trapezoidal prism 21 and travels in the trapezoidal prism 21 before reaching the light-detecting element 22.

According to the embodiment, the light as it is emitted from the light-emitting element 20 is of such an intensity that the light from the dermis reaches the light-detecting element 22. In addition, the direction along which the light emitted from the light-emitting element 20 travels is determined such that the light reflected from the dermis that reaches the light-detecting element 22 is of a maximum level of intensity. Consequently, the trapezoidal prism 21 is used to determine the direction of travel of the light emitted from the light-emitting element 20.

Since the light reflected from the dermis is used to estimate the concentration of the biological component, the amount of electric power consumed by the light-emitting element 20 is relatively low according to the embodiment.

The dermis contains arterioles, venules, and capillaries. Therefore, the dermis allows glucose to have a high degree of tissue penetration. As glucose is carried by blood in human bodies, it is contained at a high level in the dermis as well as the arteries and the veins. Part of glucose binds to hemoglobin.

FIG. 7 illustrates optical absorption spectrums of oxygenated hemoglobin (HbO₂) and reduced hemoglobin (Hb). In FIG. 7, NIR represents a near-infrared light wavelength range of 780 to 2500 nm, and RE a red light wavelength range of 610 to 780 nm.

As illustrated in FIG. 7, oxygenated hemoglobin absorbs more visible light except red light than near-infrared light, and reduced hemoglobin absorbs more visible light than near-infrared light. Inasmuch as both oxygenated hemoglobin and reduced hemoglobin are less liable to absorb near-infrared light, much of the light from the dermis that includes capillaries reaches the light-detecting element 22 providing near-infrared light is emitted from the light-emitting element 20.

Consequently, the output of the light-detecting element 22 is commensurate with the amount of hemoglobin in the dermis. Moreover, since the reflectance of hemoglobin with respect to near-infrared light differs depending on the amount of glucose that binds to hemoglobin, the output of the light-detecting element 22 is also commensurate with the amount of glucose in the dermis. Therefore, the controller 18 is able to estimate the concentration of glucose in the dermis from the output of the light-detecting element 22.

Glucose is contained at a high level in thick arteries and veins. However, as no thick arteries and veins exist in the dermis, it is unlikely that the output of the light-detecting element 22 will be affected by a plethysmogram due to blood vessel pulsations. Accordingly, the controller 18 is capable of obtaining an accurate and stable estimate of the concentration of glucose contained in the body of the examinee from the output of the light-detecting element 22.

The multiple light-emitting elements 20 emit light in different wavelength ranges. The multiple light-emitting elements 20 may emit such light simultaneously or in a time-division multiplex fashion. The controller 18 is able to obtain an accurate and stable estimate of the concentration of glucose contained in the body of the examinee from the output of the light-detecting element 22 on the basis of either the light simultaneously emitted from the multiple light-emitting elements 20 or the light emitted from the multiple light-emitting elements 20 in a time-division multiplex fashion.

However, the sensor device 10 may have only one light-emitting element 20.

As described above, the controller 18 of the sensor device 10 according to the embodiment estimates the concentration of glucose contained in the examinee by using the near-infrared light. However, the controller 18 may estimate the concentration of any of various other biological components in the examinee with the light-emitting element 20 that emits light other than the near-infrared light.

According to the noninvasive optical measurement technology, the accuracy of measurement varies depending on the pressing force applied to the human body. According to the embodiment, the output of the light-detecting element 22 varies depending on the pressing force that the trapezoidal prism 21 applies to the arm 50.

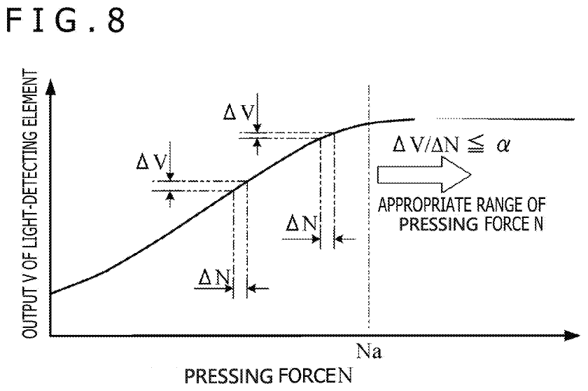

FIG. 8 illustrates a relation between the pressing force, denoted by N, and the output of the light-detecting element 22 represented by a voltage V. When the pressing force N increases, the output V of the light-detecting element 22 increases substantially linearly. When the pressing force N goes beyond a certain pressing force Na, the output V of the light-detecting element 22 stops increasing even if the pressing force N keeps increasing. In other words, the output V of the light-detecting element 22 remains stable at values of the pressing force N that are higher than the pressing force Na.

The reason why the output V of the light-detecting element 22 increases with the pressing force N is that when the pressing force N increases, the intensity of the light reflected from the dermis and detected by the light-detecting element 22 increases.

When the pressing force N is low, the trapezoidal prism 21 is not held in close contact with the arm 50. In other words, the infinitesimal spaces 51 (see FIG. 2) that are present between the trapezoidal prism 21 and the arm 50 are large. It is preferable that all the light applied from the light-emitting element 20 to the trapezoidal prism 21 travel from the first bottom surface 21a into the arm 50. However, the refractive index of the trapezoidal prism 21, the refractive index of air, and the refractive index of the skin of the arm 50 are different from each other. Therefore, in the presence of the infinitesimal spaces 51, the light tends to be refracted or reflected in directions other than desired directions by the first bottom surface 21a, i.e., the interface between the trapezoidal prism 21 and air, and the surface of the arm 50, i.e., the interface between air and the skin. The presence of the infinitesimal spaces 51 thus makes the light reflected from the dermis and detected by the light-detecting element 22 lower in intensity. However, an increase in the pressing force N reduces the infinitesimal spaces 51, increasing the light reflected from the dermis and detected by the light-detecting element 22.

Although not illustrated, when the pressing force N is very high, the blood flow in the capillaries is blocked, reducing the light reflected from the dermis and detected by the light-detecting element 22 and lowering the output V from the light-detecting element 22.

The pressing force N has an appropriate range in which the output V from the light-detecting element 22 is stable. FIG. 8 illustrates such an appropriate range indicated by the outlined allow. The pressing force Na referred to above represents the lower limit of the appropriate range for the pressing force N.

According to the embodiment, the pressing force that is applied from the trapezoidal prism 21 to the arm 50 is automatically adequately controlled using the output V from the light-detecting element 22 of the optical system for estimating the concentration of the biological component.

Specifically, the controller 18 controls the pump 16 to increase the pressing force until a change in the output V from the light-detecting element 22 becomes equal to or smaller than a first threshold value. After the change in the output V from the light-detecting element 22 has become equal to or smaller than the first threshold value, the controller 18 maintains the pressing force. Under the maintained pressing force, the controller 18 estimates the concentration of the biological component contained in human bodies, e.g., the concentration of glucose, from the output V of the light-detecting element 22.

According to the embodiment, specifically, the controller 18 estimates the concentration of the biological component with the light reflected from the dermis, by using the optical system including the multiple light-emitting elements 20, the light-detecting element 22, and the trapezoidal prism 21. In addition, the controller 18 automatically adequately controls the pressing force produced by the pump 16, by using the output V of the light-detecting element 22 of the same optical system.

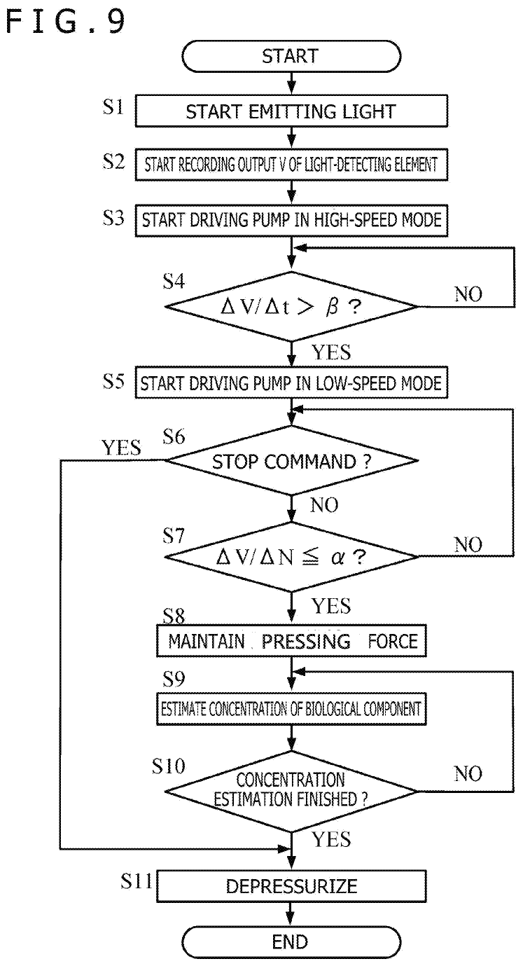

An example of operation of the sensor device 10, i.e., a process of using the sensor device 10, will be described in detail below with reference to a flowchart of FIG. 9.

The sensor device 10 starts to operate when the start button 17a of the switch unit 17 is pressed.

In step S1, the controller 18 supplies drive signals to the multiple light-emitting elements 20 to enable the multiple light-emitting elements 20 to start emitting light. In step S1, the controller 18 may supply a drive signal to a single light-emitting element 20 or drive signals to multiple light-emitting elements 20.

In step S2, the controller 18 supplies a drive signal to the light-detecting element 22 to start its photoelectric conversion process and starts to record the output V from the light-detecting element 22.

In step S3, the controller 18 supplies a drive signal to the pump 16, starting to actuate the pump 16. The actuated pump 16 starts expanding the air bag 15.

In step S3, specifically, the controller 18 starts driving the pump 16 in a high-speed mode. The high-speed mode refers to a mode of driving the pump 16 at a high speed until the trapezoidal prism 21 can press the arm 50 with a certain pressing force.

In step S4, the controller 18 determines whether a change ΔV/Δt in the output V of the light-detecting element 22 per unit time Δt is larger than a threshold value β, i.e., a second threshold value, or not. If the decision from step S4 is negative, then the processing loops back to step S4.

In other words, the controller 18 waits until the change ΔV/Δt in the output V of the light-detecting element 22 per unit time Δt reaches the threshold value β, i.e., becomes equal to or larger than the threshold value β. Stated otherwise, the pump 16 keeps being driven in the high-speed mode until the decision from step S4 becomes affirmative. Since the controller 18 continuously drives the pump 16 in the high-speed mode during an initial stage of its control over the pump 16, the air bag 15 is expanded quickly.

If the decision from step S4 is affirmative, then the processing goes to step S5. In step S5, the controller 18 starts driving the pump 16 in a low-speed mode. In the low-speed mode, the pump 16 is driven to supply air at a lower rate than the rate at which the pump 16 supplies air in the high-speed mode, thereby reducing the rate at which the air bag 15 is expanded. The low-speed mode refers to a mode of driving the pump 16 at a low speed after the trapezoidal prism 21 has pressed the arm 50 with the certain pressing force.

After the air bag 15 has started to expand in step S3, while the air bag 15 is expanded to a small degree in the initial stage, i.e., during the high-speed mode, the air bag 15 makes no or almost no contribution to the pressurization of the trapezoidal prism 21. Therefore, during the initial stage, the output V of the light-detecting element 22 does not substantially increase.

After the expansion of the air bag 15 has progressed to a certain extent, as the air bag 15 is expanded, it applies a tensile force to the belt 13, causing the support 12 to press the housing 11 toward the arm 50. Therefore, the trapezoidal prism 21 is pressed against the arm 50, increasing the pressing force N to increase the output V of the light-detecting element 22 substantially linearly, as illustrated in FIG. 8.

During the initial stage, the controller 18 drives the pump 16 in the high-speed mode to expand the air bag 15 at a high expansion rate. After the initial stage, the controller 18 drives the pump 16 in the low-speed mode to expand the air bag 15 at a low expansion rate. In the example of operation illustrated in FIG. 9, the stage until the change ΔV/Δt in the output V of the light-detecting element 22 per unit time Δt reaches the threshold value β, i.e., until the decision from step S4 becomes affirmative, represents the initial stage. The threshold value β is stored in the storage device 19. The threshold value β represents a fixed value.

In step S6 after step S5, the controller 18 determines whether it has received a stop command from the switch unit 17 or not. If the decision from step S6 is affirmative, then the processing goes to step S11.

In step S11, the controller 18 gives a command to the pump 16 to depressurize the air bag 15 in order to reduce the pressing force applied from the trapezoidal prism 21 to the arm 50. In response to the command, the pump 16 discharges air from the air bag 15. Thereafter, the processing comes to an end. If the examinee feels pain from being tightened by the belt 13 or pressed by the trapezoidal prism 21, for example, the examinee or the medical attendant presses the stop button 17b, whereupon the switch unit 17 supplies a stop command to the controller 18. Now, the decision from step S6 becomes affirmative, finishing the measurement of the concentration of the biological component.

If the decision from step S6 is negative, the processing goes to step S7. In step S7, the controller 18 determines whether or not a change in the output V of the light-detecting element 22 is equal to or smaller than a threshold value α, i.e., a first threshold value. The “change in the output V of the light-detecting element 22” in step S7 refers to a predetermined increment ΔV in the output V of the light-detecting element 22 per increment ΔN in the pressing force N, i.e., ΔV/ΔN.

The pressing force N is calculated by the controller 18 on the basis of the output from the force sensor 30. Therefore, the controller 18 is able to detect when the pressing force N has increased by the increment ΔN.

However, the “change in the output V of the light-detecting element 22” in step S7 may refer to the change ΔV/Δt in the output V of the light-detecting element 22 per unit time Δt. Alternatively, the “change in the output V of the light-detecting element 22” in step S7 may refer to a change ΔV/ΔP in the output V of the light-detecting element 22 per unit electric power quantity ΔP for driving the pump 16. If these changes are used, then as the controller 18 is not required to monitor the pressing force N, and the force sensor 30 may be dispensed with.

The threshold value α used in step S7 of the example of operation illustrated in FIG. 9 represents a fixed value. The threshold value α is stored in the storage device 19.

If the decision from step S7 is negative, then the processing loops back to step S6. Therefore, the controller 18 continuously drives the pump 16 in the low-speed mode. In other words, until the change in the output V of the light-detecting element 22 becomes equal to or smaller than the threshold value α, the controller 18 increases the pressing force N in the low-speed mode.

On the other hand, if the decision from step S7 is affirmative, i.e., the change in the output V from the light-detecting element 22 is equal to or smaller than the threshold value α, then the processing goes to step S8. In step S8, the controller 18 gives a command to the pump 16 to stop increasing the pressing force N. Stated otherwise, the pressing force N is maintained.

As illustrated in FIG. 8, after the change ΔV/ΔN in the output of the light-detecting element 22 has become equal to or smaller than the threshold value α, the pressing force N is in the appropriate range. Therefore, the pressing force N is maintained in the appropriate range in step S8.

Under the appropriately maintained pressing force, the controller 18 estimates the concentration of the biological component contained in the human body, e.g., the concentration of glucose, from the output V of the light-detecting element 22 in step S9.

Providing only one of the light-emitting element 20 has been energized prior to step S9, the controller 18 may in step S9 energize the other light-emitting element 20 and estimate the concentration of the biological component contained in the human body from the output V of the light-detecting element 22 based on the light coming from the multiple light-emitting elements 20. Alternatively, the controller may not energize the other light-emitting element 20 and may estimate the concentration of the biological component contained in the human body from the output V of the light-detecting element 22 based on the light coming from the single light-emitting element 20.

In step S10, the controller 18 determines whether the concentration estimation has been finished or not. If the concentration estimation has not been finished, i.e., the decision from step S10 is negative, then the processing goes back to step S9.

If the concentration estimation has been finished, i.e., the decision from step S10 is affirmative, then the processing proceeds to step S11.

In step S11, the controller 18 gives a command to the pump 16 to depressurize the air bag 15 in order to reduce the pressing force applied from the trapezoidal prism 21 to the arm 50. In response to the command, the pump 16 discharges air from the air bag 15.

After step S11, the processing comes to an end.

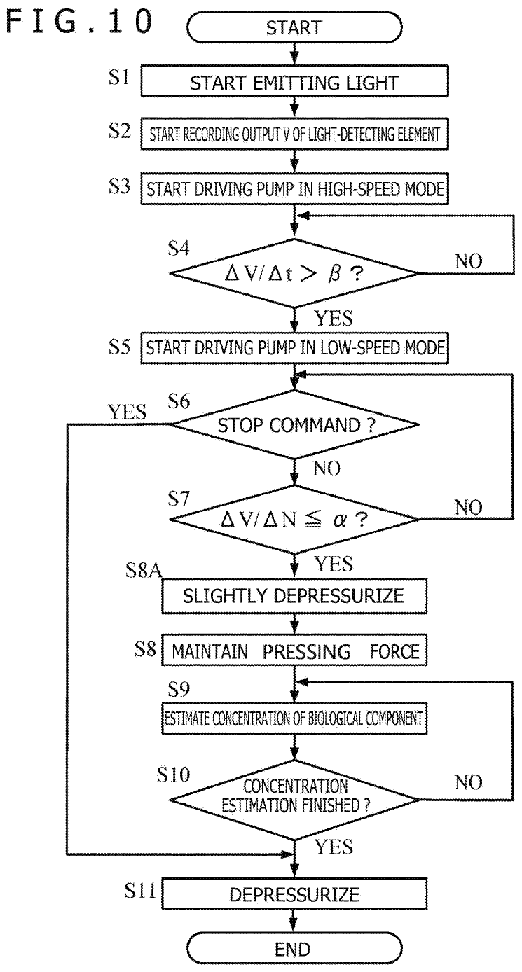

FIG. 10 illustrates a flowchart of another example of operation of the sensor device 10. The flowchart of FIG. 10 shares certain steps with the flowchart of FIG. 9. The example of operation of the sensor device 10 illustrated in FIG. 10 will be described below primarily with respect to steps that are different from the steps of the example of operation of the sensor device 10 illustrated in FIG. 9.

According to the example of operation illustrated in FIG. 10, if the decision from step S7 becomes affirmative, i.e., if the change in the output V of the light-detecting element 22 becomes equal to or smaller than the threshold value α, then the processing goes to step S8A.

In step S8A, in order to slightly reduce the pressing force N applied from the trapezoidal prism 21 to the arm 50, the controller 18 gives a command to the pump 16 to slightly depressurize the air bag 15. In response to the command, the pump 16 slightly discharges air from the air bag 15. The pump 16 may discharge a certain amount of air from the air bag 15 or may discharge air from the air bag 15 for a certain period of time. After step S8A, the processing goes to step S8 in which the controller 18 gives a command to the pump 16 to stop reducing the pressing force N, i.e., to maintain the pressing force N.

According to the example of operation illustrated in FIG. 10, therefore, the controller 18 increases the pressing force N until the change in the output V of the light-detecting element 22 becomes equal to or smaller than the threshold value α. After the change in the output V of the light-detecting element 22 has become equal to or smaller than the threshold value α, the controller 18 slightly reduces the pressing force N and thereafter maintains the pressing force N.

Although the threshold value α used in step S7 is a fixed value, a relation between the pressing force N and the output V from the light-detecting element 22 differs from examinee to examinee. FIG. 8 illustrates only an example of pressing forces N and outputs V from the light-detecting element 22. A preferable pressing force N for an examinee may possibly differ depending on the type of the examinee, e.g., the gender, race, age, and/or constitution. In other words, the preferable pressing force N for an examinee may not be preferable for other examinees.

According to the example of operation illustrated in FIG. 10, the pressing force N is increased to a somewhat high level and thereafter the air bag 15 is slightly depressurized in step S8A. Therefore, the threshold value α used in the example of operation illustrated in FIG. 10 is smaller than the threshold value α used in the example of operation illustrated in FIG. 9. The amount of air discharged from the air bag 15 or the period of time for which air is discharged from the air bag 15 that determines the extent to which the air bag 15 is to be depressurized in step S8A may be of a fixed value or may be of a value that differs depending on the type of the examinee. The value that represents the amount of air discharged from the air bag 15 or the period of time for which air is discharged from the air bag 15 is stored in the storage device 19.

According to the example of operation illustrated in FIG. 9, the controller 18 may determine the threshold value α based on the type of the examinee, e.g., the gender, race, age, and/or constitution prior to step S1. A table or formula representing a relation between the types of examinees and threshold values xx is stored in the storage device 19. The controller 18 displays on a display device, not illustrated, a message for prompting the examinee or the medical attendant to enter the type of the examinee. When the examinee or the medical attendant enters the type of the examinee, the controller 18 determines a threshold value α by using the table or the formula and stores the determined threshold value α in the storage device 19.

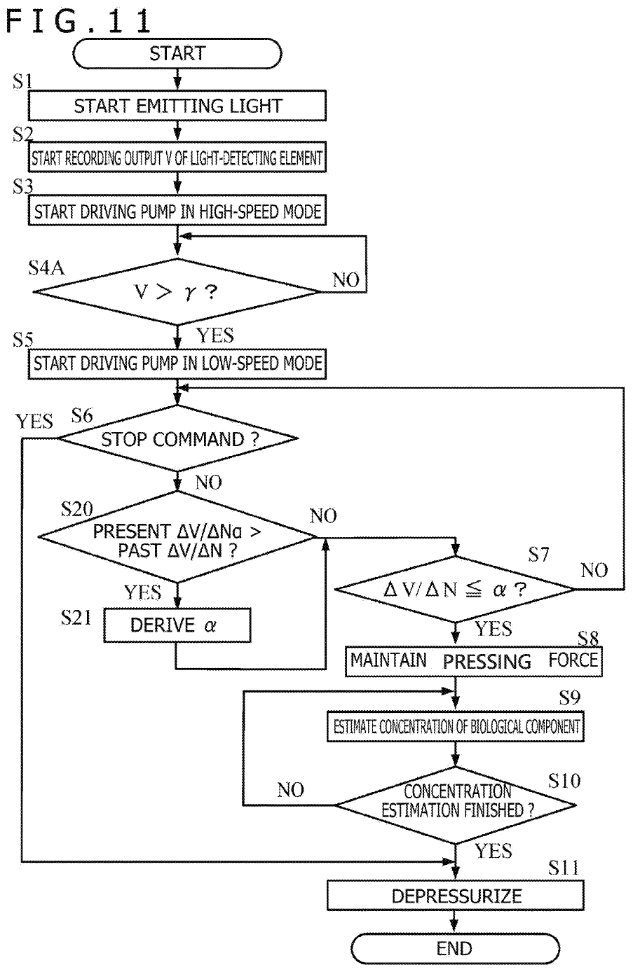

FIG. 11 illustrates a flowchart of still another example of operation of the sensor device 10. The flowchart of FIG. 11 shares certain steps with the flowchart of FIG. 9. The example of operation of the sensor device 10 illustrated in FIG. 11 will be described below primarily with respect to steps that are different from the steps of the example of operation of the sensor device 10 illustrated in FIG. 9.

According to the example of operation illustrated in FIG. 11, after step S3, step S4A is carried out instead of step S4. In step S4A, the controller 18 determines whether the output V of the light-detecting element 22 is larger than a threshold value γ, i.e., a third threshold value, or not. If the decision from step S4A is negative, then the processing loops back to step S4A.

In other words, the controller 18 waits until the output V1 of the light-detecting element 22 reaches the threshold value γ, i.e., becomes equal to or larger than the threshold value γ. Stated otherwise, the pump 16 keeps being driven in the high-speed mode until the decision from step S4A becomes affirmative. Since the controller 18 continuously drives the pump 16 in the high-speed mode during an initial stage of its control over the pump 16, the air bag 15 is expanded quickly.

If the decision from step S4A is affirmative, then the processing goes to step S5. In step S5, the controller 18 starts driving the pump 16 in a low-speed mode. In the low-speed mode, the pump 16 is driven to supply air at a lower rate than the rate at which the pump 16 supplies air in the high-speed mode, thereby reducing the rate at which the air bag 15 is expanded.

During the initial stage, the controller 18 drives the pump 16 in the high-speed mode to expand the air bag 15 at a high expansion rate. After the initial stage, the controller 18 drives the pump 16 in the low-speed mode to expand the air bag 15 at a low expansion rate. In the example of operation illustrated in FIG. 11, the stage until the output V of the light-detecting element 22 reaches the threshold value γ, i.e., until the decision from step S4A becomes affirmative, represents the initial stage. The threshold value γ represents a fixed value. The threshold value γ is stored in the storage device 19.

According to the example of operation illustrated in FIG. 11, after step S6, the processing goes to step S20. In step S20, the controller 18 determines whether a change in the output V of the light-detecting element 22 in the present is larger than the change in the output V in the past or not. The “change in the present output V of the light-detecting element 22” refers to an increment ΔV in the output V of the light-detecting element 22 per predetermined increment ΔN in the pressing force N, i.e., ΔV/ΔN.

However, the “change in the output V of the light-detecting element 22” in step S20 may refer to the change ΔV/Δt in the output V of the light-detecting element 22 per unit time Δt. Alternatively, the “change in the output V of the light-detecting element 22” in step S20 may refer to a change ΔV/ΔP in the output V of the light-detecting element 22 per unit electric power quantity ΔP for driving the pump 16. If these changes are used, then as the controller 18 is not required to monitor the pressing force N, the force sensor 30 may be dispensed with.

For the determining process in step S20, the controller 18 stores the change in the output V of the light-detecting element 22 in the storage device 19.

If the decision from step S20 is affirmative, the processing goes to step S21. Providing no change in the output V of the light-detecting element 22 in the past is stored in the storage device 19, i.e., providing step S20 is carried out for the first time after the pump 16 has started to be driven, the decision from step S20 is affirmative.

In step S21, the controller 18 multiplies the change in the output V in the present by a predetermined coefficient x to derive a threshold value α and stores the derived threshold value α in the storage device 19. If the “change in the output V of the light-detecting element 22” represents ΔV/ΔN, then a threshold value α is derived according to the following equation:

α=(ΔV/ΔN)x

Then, the processing goes to step S7. In step S7, the controller 18 compares the change in the output V of the light-detecting element 22 with the threshold value α derived in step S21 to determine whether or not the change in the output V is equal to or smaller than the threshold value α. If the decision from step S7 is negative, then the processing loops back to step S6. If the decision from step S20 is negative, the processing goes to step S7.

Each time the controller 18 decides that the change in the output V in the present is larger than the change in the output V in the past by repeating the above control loop, the controller 18 updates the threshold value α stored in the storage device 19. As a result, the controller 18 derives a threshold value α by multiplying the maximum value of the change in the output V of the light-detecting element 22 by the predetermined coefficient x.

As described above, the relation between the pressing force N and the output V from the light-detecting element 22 differs from examinee to examinee. According to the example of operation illustrated in FIG. 11, since the threshold value α is derived on the basis of the maximum value of the change in the output V of the light-detecting element 22, the pressing force N can adequately be controlled depending on the examinee. Furthermore, as a threshold value α is derived while the pressing force N is being increased, i.e., during the above control loop, the threshold value α is derived in the process of operation for measuring the concentration of the biological component. Inasmuch as there is no need to perform a process of deriving a threshold value α separately from the process of operation for measuring the concentration of the biological component, the time efficiency of the operation of the controller 18 is high.

Steps S20 and S21 may also be carried out in the example of operation illustrated in FIG. 10.

While the preferred embodiment and modifications according to the present disclosure have been described in detail above, it will be obvious to those skilled in the art that various changes, modifications, and revisions can be made therein without departing from the scope of the disclosure. Such changes, modifications, and revisions should fall within the scope of the disclosure.

For example, step S4 illustrated in FIG. 10 or step S4A illustrated in FIG. 11 may not be carried out, and the controller 18 may drive the pressing mechanism in the high-speed mode for a predetermined period of time representing the initial stage, and then, after elapse of a predetermined period of time, may start the low-speed mode in step S5.

The sensor device may have another pressing mechanism instead of the air bag 15 and the pump 16. The other pressing mechanism may include a hydraulic mechanism, a linear actuator, or a cam mechanism, for example.

The present disclosure contains subject matter related to that disclosed in Japanese Priority Patent Application JP 2025-042961 filed in the Japan Patent Office on Mar. 17, 2025, the entire content of which is hereby incorporated by reference.

What is claimed is:

1. A sensor device comprising:
a light-emitting element;
a prism that is of a trapezoidal contour as viewed along a direction and has a first bottom surface for contact with a surface of a human body, a slanted surface to which light emitted from the light-emitting element is applied, and a second bottom surface lying parallel to the first bottom surface, the prism refracting light emitted from the light-emitting element toward the human body;
a light-detecting element that detects light that has been reflected by a region in the human body, transmitted through the prism, and emitted from the second bottom surface;
an estimating section that estimates a concentration of a biological component contained in the human body from an output from the light-detecting element;
a pressing mechanism that presses the prism against the human body; and
a controlling section that controls the pressing mechanism, wherein,
when a change in the output from the light-detecting element exceeds a first threshold value, the controlling section controls the pressing mechanism to increase its pressing force until the change in the output from the light-detecting element becomes equal to or smaller than the first threshold value, and after the change in the output from the light-detecting element has become equal to or smaller than the first threshold value, the controlling section maintains the pressing force, and
the estimating section estimates the concentration of the biological component contained in the human body from the output from the light-detecting element under the maintained pressing force.

2. The sensor device according to claim 1, wherein
the controlling section controls the pressing mechanism to reduce the pressing force and then maintain the pressing force after the change in the output from the light-detecting element has become equal to or smaller than the first threshold value.

3. The sensor device according to claim 1, wherein
the controlling section derives the first threshold value by multiplying a maximum value of the change in the output from the light-detecting element by a predetermined coefficient.

4. The sensor device according to claim 3, wherein
the controlling section derives the first threshold value while controlling the pressing mechanism to increase the pressing force.

5. The sensor device according to claim 1, wherein
the controlling section
drives the pressing mechanism in a first mode during an initial stage of its control over the pressing mechanism, and,
after the initial stage, drives the pressing mechanism in a second mode that is of a lower rate than the first mode to increase the pressing force from the pressing mechanism until the change in the output from the light-detecting element becomes equal to or smaller than the first threshold value.

6. The sensor device according to claim 5, wherein
the controlling section
drives the pressing mechanism in the first mode until a change in the output from the light-detecting element per unit time becomes equal to or larger than a second threshold value, and,
after the change in the output from the light-detecting element per unit time has become equal to or larger than the second threshold value, drives the pressing mechanism in the second mode.

7. The sensor device according to claim 5, wherein
the controlling section
drives the pressing mechanism in the first mode until the output from the light-detecting element becomes equal to or larger than a third threshold value, and,
after the output from the light-detecting element has become equal to or larger than the third threshold value, drives the pressing mechanism in the second mode.

8. The sensor device according to claim 1, wherein
the light-emitting element emits near-infrared light in a wavelength range of 780 to 2500 nm.
